(19) United States
(12) Patent Application Publication
(10) Pub. No.: US 2021/0324073 A1
(43) Pub. Date: Oct. 21, 2021

(54) IgA mediated killing of aberrant cells by CD47- SIRPalpha checkpoint inhibition of neutrophils

(71) Applicant: UMC Utrecht Holding B.V., Utrecht (NL)

(72) Inventor: Jeannette Henrica Wilhelmina LEUSEN, Utrecht (NL)

(21) Appl. No.: 17/242,954

(22) Filed: Apr. 28, 2021

Related U.S. Application Data
(63) Continuation of application No. PCT/NL2019/050712, filed on Oct. 29, 2019.

(30) Foreign Application Priority Data
Oct. 29, 2018 (EP) 18203183.1

(51) Int. Cl. C07K 16/28; A61P 35/00

(52) U.S. Cl. CPC C07K 16/283; C07K 16/2896; A61K 2039/507; C07K 2317/76; A61P 35/00

(57) ABSTRACT

The invention provides means and methods for stimulating neutrophil-mediated killing of CD47 expressing cells. Methods may include contacting neutrophils with cells that express CD47 and another extracellular membrane-bound antigen in the presence of a first and a second binding moiety, wherein said first binding moiety specifically binds a myeloid IgA receptor (CD89) and said antigen, and wherein said second binding moiety specifically binds CD47 and/or SIRPα and blocks CD47 mediated signaling of SIRPα in said neutrophil.

### CROSS-REFERENCE

This application is a Continuation Application of International Patent Application PCT/NL2019/050712, filed Oct. 29, 2019, which claims the benefit of European Patent Application EP 18203183.1, filed Oct. 29, 2018; each of which are each incorporated herein by reference in their entirety.

### SEQUENCE LISTING

The instant application contains a Sequence Listing which has been submitted electronically in ASCII format and is hereby incorporated by reference in its entirety. Said ASCII copy, created on Apr. 27, 2021, is named 55207-712_301_SL.txt and is 18,995 bytes in size.

The invention relates to the field of antibodies, in particular to the field of therapeutic antibodies. The invention also relates to the field of immunotherapy, in particular to reducing immune response inhibitory processes in cancer.

Cancer treatments have evolved considerably in recent years. Many experimental and regular treatments presently include the administration of one or more antibodies directed towards tumor cells and/or immune cells. Such antibodies are lytic by themselves, because of an antibody drug conjugate, or because the immune system is stimulated or less inhibited to act against the tumor cells. Antibody treatments in cancer include trastuzumab, cetuximab, and rituximab, which target HER2/neu, EGFR, or CD20, respectively. All FDA-approved antibody drugs are IgG isotypes. However, antibodies of the IgA isotype have been shown to be effective against tumors in vitro and in vivo (e.g. Boross et al, 2013 EMBO Mol Med 5: 1213-26). IgG is generally associated with the blood stream, and IgA is mostly known for its secretory aspect and the resulting presence at mucosal sites in its dimeric form. IgA is comprised of two subclasses, IgA1 and IgA2. Both IgA types bind with similar affinity to the myeloid IgA receptor (FcαRI, CD89). The secretory form is not bound by the receptor.

Immune-mediated effects of antibodies include cytotoxicity induced by complement activation, antibody-dependent cellular phagocytosis (ADCP) and antibody-dependent cellular cytotoxicity (ADCC). ADCC can be mediated through activation of different Fc-receptor expressing cells, including natural killer (NK) cells, macrophages and neutrophils. Macrophages and neutrophils express the CD89 receptor that binds IgA antibodies, and can kill tumor cells by ADCP or ADCC as demonstrated. Antibodies of the IgA class have shown results in inducing ADCC of various tumor targets, including HER2/neu⁺- and EGFR⁺-carcinomas and CD20-positive lymphomas.

Various checkpoint inhibition molecules are presently known. Most of them are studied to see if they can function in a reversal of immune response inhibition therapy. Inhibitory receptor signal regulatory protein alpha (SIRPα) or its ligand CD47 was effective in pre-clinical models when combined with IgG₁ and IgG₂ anti-cancer therapies (Zhao, Proc Natl Acad Sci USA. 2011; 108(45):18342-7, Matlung et al., Immunol Rev. 2017; 276(1):145-64; Chao et al., Cell. 2010; 142(5):699-713. Weiskopf et al., Science. 2013; 341(6141):88-91; Treffers et al Eur J Immunol. 2018, 48(2):344-354, Rosner et al, Mol Cancer Ther. 2018. doi: 10.1158/1535-7163.MCT-18-0341. [Epub ahead of print]. CD47-SIRPα interaction blocking agents are currently being tested in clinical trials for hematological and solid cancers (www.clinicaltrials.gov identifiers: NCT02216409; NCT02678338, NCT02641002; NCT02367196, NCT02890368; NCT02663518, NCT02953509).

SIRPα is present on myeloid cells. The ligand CD47 is expressed by many cells and is often said to act as a ‘don't eat me’ signal. It is frequently over-expressed on cancer cells. In some pre-clinical models, CD47-SIRPα blockade enhanced cancer immunotherapies when combined with IgG mAbs targeting different tumor antigens, such as cetuximab, trastuzumab and rituximab. A combination of an IgA antibody having the variable region of rituximab with an anti-SIRPα antibody (KWAR23) is described in WO2015/138600.

In the present invention it is shown that CD47-SIRPα checkpoint inhibition strongly enhances the effect of the IgA antibodies in vivo. We show that blocking CD47-SIRPα interactions in in vitro experiments and in both xenogeneic and syngeneic in vivo mouse models leads to an enhancement of IgA-based anti-cancer therapies. In the syngeneic mouse model, we demonstrate a prominent increase in neutrophil influx when IgA therapy is combined with SIRPα block, and that these neutrophils are essential for the clearance of the tumor cells, since depletion of these neutrophils abrogates the therapy. This is a unique feature for IgA, because an IgG therapeutic molecule does not show this strong recruitment of neutrophils, nor the strong enhancement of tumor kill in the presence of CD47-SIRPα checkpoint blockade.

### SUMMARY OF THE INVENTION

The invention provides a method of stimulating neutrophil-mediated killing of CD47 expressing cells comprising contacting neutrophils with cells that express CD47 and another extracellular membrane-bound antigen in the presence of a first and a second binding moiety, wherein said first binding moiety specifically binds a myeloid IgA receptor (CD89) and said antigen, and wherein said second binding moiety specifically binds CD47 and/or SIRPα and blocks CD47 mediated signaling of SIRPα in said neutrophils.

The invention also provides a first binding moiety and a second binding moiety, wherein said first binding moiety specifically binds CD89 and another extracellular membrane-bound antigen, and wherein said second binding moiety specifically binds CD47 and/or SIRPα and blocks CD47 mediated signaling of SIRPα.

The invention further provides a method of increasing the influx of neutrophils in a cancer of an individual, the method comprising administering a first binding moiety and a second binding moiety to the individual in need thereof, wherein said first binding moiety specifically binds CD89 and an extracellular membrane-bound antigen on cells in said caner, and wherein said second binding moiety specifically binds CD47 and/or SIRPα and blocks CD47 mediated signaling of SIRPα.

The invention further provides a first binding moiety and a second binding moiety for use in the treatment of an individual that has cancer, wherein said first binding moiety specifically binds CD89 and an extracellular membrane-bound antigen, and wherein said second binding moiety specifically binds CD47 and/or SIRPα and blocks CD47 mediated signaling of SIRPα.

Said tumor preferably has tumor cells, tumor stromal cells and/or Tregulator cells. The first binding moiety is preferably an IgA antibody that binds an extracellular membrane-bound antigen on tumor cells of said tumor. The extracellular membrane-bound antigen is preferably a further extracellular membrane-bound antigen that is not an IgA receptor. The other extracellular membrane-bound antigen is preferably bound with a variable domain of the antibody. The tumor is preferably sensitive to neutrophil mediated ADCC or trogocytosis. The extracellular membrane-bound antigen on the cells is preferably selected from the group consisting of: CD19, CD21, CD22, CD24, CD27, CD30, CD33, CD38, CD44, CD52, CD56, CD64, CD70, CD96, CD97, CD99, CD115, CD117, CD123, mesothelin, Chondroitin Sulfate Proteoglycan 4 (CSPG4), PD-L1 (CD274), Her2/neu (CD340), Her3, EGFR, PDGFR, SLAMF7, VEGFR1, VEGFR2, DR5, TF, GD2, GD3, PTHR2, CTLA4 or CD2.

The invention further provides nucleic acid molecules that code for the first and second binding moiety.

The invention further provides cells that comprise nucleic acid that codes for and expresses the first binding moiety, the second binding moiety or both.

The first binding moiety preferably binds CD89 and another extracellular membrane-bound antigen. The first binding moiety is preferably an antibody that binds the myeloid IgA receptor (CD89). The first binding moiety, first antibody preferably comprises a constant region that binds CD89. The constant region is preferably an IgA constant region. The first antibody preferably comprises an IgA CH1, CH2, CH3 and hinge region. The other extracellular membrane-bound antigen is preferably bound by one or more of the variable regions of the first antibody.

The second binding moiety preferably specifically binds CD47 and/or SIRPα and blocks CD47 mediated signaling of SIRPα in said neutrophil.

The invention further provides a method of treatment of an individual that has a tumor, the method comprising administering a first binding moiety and a second binding moiety to the individual in need thereof, wherein said first binding moiety specifically binds CD89 and an extracellular membrane-bound antigen on cells in said tumor, and wherein said second binding moiety specifically binds CD47 and/or SIRPα and blocks CD47 mediated signaling of SIRPα.

Also provided is a method for targeting neutrophils to cells in an individual, the method comprising administering a first binding moiety and a second binding moiety to the individual in need thereof, wherein said first binding moiety specifically binds CD89 and an another extracellular membrane-bound antigen on cells, and wherein said second binding moiety specifically binds CD47 and/or SIRPα and blocks CD47 mediated signaling of SIRPα.

### DETAILED DESCRIPTION OF THE INVENTION

The name neutrophil derives from staining characteristics on hematoxylin and eosin (H&E) histological or cytological preparations. Whereas basophilic white blood cells stain dark blue and eosinophilic white blood cells stain bright red, neutrophils stain a neutral pink. Normally, neutrophils contain a nucleus divided into 2-5 lobes. Neutrophils are a type of phagocyte and are normally found in the bloodstream. During the beginning (acute) phase of inflammation, particularly as a result of bacterial infection, environmental exposure, and some cancers, neutrophils are one of the first-responders of inflammatory cells to migrate towards the site of inflammation. They migrate through the blood vessels, then through interstitial tissue, following chemical signals such as Interleukin-8 (IL-8), C5a, fMLP, Leukotriene B4 and H2O2 in a process called chemotaxis.

A method of stimulating neutrophil-mediated killing of CD47 expressing cells is provided comprising contacting neutrophils with cells that express CD47 and another extracellular membrane-bound antigen in the presence of a first and a second binding moiety, wherein said first binding moiety specifically binds a myeloid IgA receptor (CD89) and said antigen, and wherein said second binding moiety specifically binds CD47 and/or SIRPα and blocks CD47 mediated signaling of SIRPα in said neutrophil.

The neutrophils are contacted with cells that express CD47 and another extracellular membrane-bound antigen on the cell. The other extracellular membrane-bound antigen can be used to select the particular cell type that is to be killed by the neutrophils, to select the target on the cell that is used to direct the neutrophil or for another reason. Some reasons for target selection being that some targets or more abundant, more amiable for targeting, more accessible, and/or have functionality that may be enhanced or inhibited by the binding of the binding moiety or a combination of binding moieties.

The other extracellular membrane-bound antigen is preferably an antigen that is present on tumor cells. Such an antigen is further referred to as a tumor-antigen. The tumor-antigen may be tumor selective in the sense that the antigen is, in adults, only expressed significantly on tumor cells. Often the tumor antigen is not tumor-selective but chosen for other reasons. For instance but not limited to being in a pathway that is dysfunctional in the cell. In non-limiting cases the tumor-antigen is one that is over-expressed by the cell. The tumor antigen is preferably an antigen selected from CD19, CD21, CD22, CD24, CD27, CD30, CD33, CD38, CD44, CD52, CD56, CD64, CD70, CD96, CD97, CD99, CD115, CD117, CD123, mesothelin, Chondroitin Sulfate Proteoglycan 4 (CSPG4), PD-L1 (CD274), Her2/neu (CD340), Her3, EGFR, PDGFR, SLAMF7, VEGFR1, VEGFR2, DR5, TF, GD2, GD3 or PTHR2.

In one embodiment the tumor antigen the tumor-antigen is CD20.

The means, methods and uses of the invention in some embodiments do not include a first binding moiety that specifically binds CD20.

The means, methods and uses of the invention in some embodiments do not include a first binding moiety that specifically binds CD89 and CD20.

The means, methods and uses of the invention in some embodiments do not include a first binding moiety that specifically binds CD89 and CD20 and a second binding moiety that specifically binds SIRPα.

The means, methods and uses of the invention in some embodiments do not include a first binding moiety that specifically binds CD89 and CD20 and a second binding moiety that specifically binds SIRPα, wherein said first binding moiety comprises the CDR1, CDR2, and CDR3 regions of rituximab.

The means, methods and uses of the invention in some embodiments do not include a first antibody that specifically binds CD89 and CD20 and a second antibody that specifically binds SIRPα.

The means, methods and uses of the invention in some embodiments do not include a first IgA antibody that specifically binds CD20 and a second antibody that specifically binds SIRPα, wherein said first antibody preferably comprises the CDR1, CDR2, and CDR3 regions of rituximab.

The other extracellular membrane-bound antigen is preferably an antigen that is present on Tregulator cells (Tregs). The regulatory T cells, also known as suppressor T cells, are a subpopulation of T cells that modulate the immune system, maintain tolerance to self-antigens, and prevent autoimmune disease. Tregs are immunosuppressive and generally suppress or downregulate induction and proliferation of effector T cells. Such cells are often found in tumors where they are thought to inhibit or decrease an immune response of the host to the tumor cells. In the present invention the other extracellular membrane-bound antigen is preferably an antigen on Tregs (also referred to as a Treg antigen), preferably but not limited to CTLA4 or CD25. Killing of Tregs in the tumor enhances an immune response in the tumor.

The other extracellular membrane-bound antigen is preferably an antigen expressed on tumor stromal cells. Stromal cells are connective tissue cells of any organ, for example in the uterine mucosa (endometrium), prostate, bone marrow, lymph node and the ovary. They are cells that support the function of the parenchymal cells of that organ. The most common stromal cells include fibroblasts and pericytes. The interaction between stromal cells and tumor cells is known to play a major role in cancer growth and progression. It is believed that many tumors cannot grow if not also stromal cells grow. Certain types of skin cancers (basal cell carcinomas) cannot spread throughout the body because the cancer cells require nearby stromal cells to continue their division. The loss of these stromal growth factors when the cancer moves throughout the body prevents the cancer from invading other organs. Neutrophil induced cell kill of stroma cells can decrease tumor growth.

The first binding moiety binds CD89 and said other extracellular membrane-bound antigen, thereby linking the CD89 expressing cells and the cells expressing the other extracellular membrane-bound antigen together. Binding of the binding moiety to CD89 can activate cell kill activity in the neutrophil. The cell kill activity may comprise ADCC activity, ADCP activity or combination thereof or other cell kill activity of the neutrophil.

The second binding moiety binds CD47 that is expressed by cells. It is typically also expressed by the cell that expresses the other extracellular membrane-bound antigen. CD47 expressed by the cell is thought to be able to interact with SIRPα on the neutrophil and decrease an immune response that would otherwise be expressed by the SIRPα expressing cell. SIRPα is among others expressed on neutrophils.

When herein reference is made to a binding moiety such as but not limited to an antibody binding to an antigen, it is intended to specify the binding capacity of the binding agent. It does typically not mean that the binding agent is actually bound by the antigen in such cases. It also refers to the binding moiety when it is not associated or bound to the antigen. A binding moiety such as an antibody binds antigen by binding an epitope on the antigen. A binding moiety such as an antibody is said to bind to the antigen if it binds an epitope on the antigen and not, or at least much less to other epitopes. For instance, a CD47 specific antibody binds to CD47 with a KD of at least 10e-6, preferably 10-e7 or less. It binds at least 100 fold less to another extracellular antigen present on adult cells.

The second binding moiety specifically binds CD47 and/or SIRPα and blocks CD47 mediated signaling of SIRPα in the neutrophils. The CD47/SIRPα signaling axis and its utility in clinical settings is recently reviewed in Matlung et al., Immunol Rev. 2017; 276(1):145-64. Matlung et al., describe various antibodies that bind CD47 or SIRPα and that block the signaling of the molecules. The blocking capacity is, of course, compared to otherwise the same conditions but in the absence of the blocking molecule. A preferred SIRPα signaling that is blocked is signaling that induces the immune response dampening effect. Preferred CD47 binding antibodies are antibody C47A8-CQ described in EP2992089; 5A3-M5 described in US20140303354; and 2.3D11 described in US2018201677, which are incorporated by reference herein for specification of preferred CD47 binding antibodies and that block CD47-SIRPα signaling. Preferred SIRPα binding antibodies are described in WO2017178653 which is incorporated by reference herein for specification of preferred SIRPα binding antibodies. The preferred antibody therein blocks CD47-SIRPα signaling.

Stimulation of neutrophil-mediated killing of CD47 expressing cells can be measured by measuring the killing in the presence and absence of the binding moieties of the invention. An additional cell killing in the presence is considered to be neutrophil mediated cell killing. The effect is typically measured in an in vitro system but can also, at least semi quantitatively be measured in vivo.

A binding moiety as defined herein is a proteinaceous binding moiety. The binding moiety is typically a peptide, a cyclic or bicyclic peptide of up to and including 20 amino acids or a polypeptide having more than 20 amino acid residues. The art knows many proteinaceous binding molecules. Often these include one or more complete or derivative antibody variable domains. Non-limiting examples are single chain Fv-fragments, monobodies, VHH, Fab-fragments. Derivative variable domains can be artificial or naturally evolved derivatives both belonging to the class of proteins that have the immunoglobulin fold. Examples of non-immunoglobulin fold containing proteinaceous binding moieties are the avimers initially developed by Amgen.

A binding moiety as described herein is preferably an antibody. An antibody, also known as an immunoglobulin (Ig), is a large, typically Y-shaped protein. An antibody interacts with various components of the immune system. Some of the interactions are mediated by its Fc region (located at the base of the “Y”), which contains site(s) involved in these interactions.

Antibodies are proteins belonging to the immunoglobulin superfamily. They typically have two heavy chains and two light chains. There are several different types of antibody heavy chains that define the five different types of crystallisable fragments (Fc) that may be attached to the antigen-binding fragments. The five different types of Fc regions allow antibodies to be grouped into five isotypes. An Fc region of a particular antibody isotype is able to bind to its specific Fc receptor (FcR) thus allowing the antigen-antibody complex to mediate different roles depending on which FcR it binds. The ability of an IgG antibody to bind to its corresponding FcR is modulated by the presence/absence of interaction sites and the structure of the glycan(s) (if any) present at sites within its Fc region. The ability of antibodies to bind to FcRs helps to direct the appropriate immune response for each different type of foreign object they encounter.

Though the general structure of all antibodies is similar, a region at the tip of the protein is extremely variable, allowing millions of antibodies with slightly different tip structures, or antigen-binding sites, to exist. This region is known as the hypervariable region. The enormous diversity of antigen binding by antibodies is largely defined by the hypervariable region and the variable domain containing the hypervariable region.

An antibody of the invention is typically a full-length antibody. The term ‘full length antibody’ is defined as comprising an essentially complete immunoglobulin molecule, which however does not necessarily have all functions of an intact immunoglobulin. For the avoidance of doubt, a full length antibody has two heavy and two light chains. Each chain contains constant (C) and variable (V) regions. A heavy chain of a full length antibody typically comprises a CH1, a CH2, a CH3, a VH region and a hinge region. A light chain of a full length antibody typically comprises a CL region and a VL region.

An antibody binds to antigen via the variable region domains contained in the Fab portion. An antibody variable domain comprises a heavy chain variable region and a light chain variable region. Full length antibodies according to the invention encompass heavy and light chains wherein mutations may be present that provide desired characteristics. Full length antibodies should not have deletions of substantial portions of any of the regions. However, IgG or IgA molecules wherein one or several amino acid residues are substituted, inserted, deleted or a combination thereof, without essentially altering the antigen binding characteristics of the resulting antibody, are embraced within the term “full length” antibody. For instance, a ‘full length” antibody can have a substitution, insertion, deletion or a combination thereof, of between 1 and 10 (inclusive) amino acid residues, preferably in non-CDR regions, wherein the deleted amino acids are not essential for the antigen binding specificity of the antibody.

The first binding moiety is preferably an IgA antibody. The antibody preferably specifically binds a myeloid IgA receptor (CD89) via the constant region of the antibody and said antigen via one or more of the variable domains.

IgA has two subclasses (IgA1 and IgA2) and can be produced as a monomeric as well as a dimeric form. The antibody in the present invention is preferably a monomeric antibody. The IgA elements in an antibody of the invention are preferably human IgA elements. An IgA element can be an IgA1 element or an IgA2 element. IgA elements in an antibody of the invention can be all IgA1 elements or all IgA2 elements or a combination of IgA1 and IgA2 elements. An IgA element is preferably a human IgA element. Preferably all IgA element in the antibody are human IgA elements. The IgA elements can be IgA1 elements, preferably human IgA1 elements. The IgA elements can also be IgA2, preferably IgA2m(1) elements, preferably human IgA1 elements. It is preferred that the CH1 domain, CH3 domain or combination thereof is an IgA CH1 domain, an IgA CH3 domain or a combination thereof. It is preferred that the IgA CH1 domain and/or hinge region is a human IgA CH1 domain and/or human IgA hinge region. Said human IgA CH1 domain and/or human IgA hinge region is preferably an human IgA1 CH1 domain or human IgA1 hinge region. Said human IgA CH1 domain and/or human IgA hinge region is preferably an human IgA2m(1) CH1 domain or human IgA2m(1) hinge region. The constant domains and hinge region of the antibody are preferably human constant regions and hinge region, preferably of a human IgA antibody. The constant domains and hinge region of the antibody are preferably human IgA1 or human IgA2m(1) constant domains and hinge region.

A human constant region can have 0-15 amino acid changes with respect to a human allele as found in nature. An amino acid change may be introduced for various reasons. Non-limiting examples include but are not limited to improving production or homogeneity of the antibody, adapting half-life in the circulation, stability of the HC/LC combination, optimizing glycosylation, adjusting dimerization or complex formation, adjusting ADCC activity. A human constant region can have 0; 1; 2; 3; 4; 5; 6; 7; 8; 9; 10; 11; 12; 13; 14; and 15 amino acid changes with respect to a human allele as found in nature. The changed amino acid is preferably one chosen from an amino acid at a corresponding position of a different isotype.

In one embodiment the constant regions of the heavy chain are IgA2 constant regions, preferably human IgA2 constant regions, preferably human IgA2m(1). In one aspect the human constant region is a mutated IgA2m(1) sequence.

In one embodiment the antibody comprises the constant regions of an IgA2m(1) sequence, preferably with at least one and preferably at least 2; 3; 4; 5; and preferably at least 7 of the following mutations: N166G; P221R; N337T; I338L; T339S; C331S; and mutation of the C-terminal amino acid sequence which is a human IgA2m(1) antibody is “ . . . VDGTCY” into “ . . . VDGT. FIG. 3 shows the sequence of human IgA1; IgA2m(1) and a preferred mutated IgA2m(1) sequence (hIgA2.0).

In an alternative embodiment the antibody comprises the constant regions of an hIgA2.0 region, wherein 3-20 of the C-terminal amino acids are deleted, thus creating an IgA3.0 constant region. FIG. 3E shows the sequence of the preferred hIgA2.0 constant region, wherein the C-terminal amino acids that can be deleted to create the IgA3.0 constant region are underlined.

Although IgA is known as a mucosal antibody, in its monomeric form it is the second class of antibody present in the human serum. In previous studies we have shown that an anti-tumor antibody IgA can be effective in vitro. The anti-tumor mechanism is different and mainly through the recruitment of neutrophils, the most abundant type of leucocytes. Also in vivo IgA can be efficacious as a therapeutic antibody. An IgA molecule can but does not have to have an average relatively short half-life. The art knows various methods to increase the half-life of an IgA antibody in vivo. One is to effect different glycosylation. Another is to include binding aspects that facilitate binding to the neonatal Fc receptor, FcRn. In one aspect the present invention provides specific IgA antibodies by providing them with adapted glycosylation and/or targeting of the IgA to FcRn indirectly with e.g. and albumin binding domain (ABD) (Meyer et al., 2016 MAbs Vol 8: pp 87-98), or directly by an FcRn targeting moiety, such as the DIII domain of albumin. The IgA can thus be an adapted IgA antibody with one or more mutations and/or a chimer of the constant region of two or more IgA molecules.

The second binding moiety is preferably an antibody. The constant region of this antibody is preferably modified such that it does not mediated effector function. The antibody is preferably an IgG4 or an effector function deficient modified IgG1, IgG2 or IgG3.

The cells of the tumor and the tumors are preferably neoplastic cells or neoplasms. A neoplasm is an abnormal growth of tissue and when it also forms a mass is commonly referred to as a tumor. A neoplasm in the present invention typically forms a mass. A neoplastic cell is a cell from a neoplasm that has formed a mass. The World Health Organization (WHO) classifies neoplasms into four main groups: benign neoplasms, in situ neoplasms, malignant neoplasms, and neoplasms of uncertain or unknown behavior. Malignant neoplasms are also simply known as cancers. The cancer is preferably an adenocarcinoma. Preferred cancers are colorectal cancer; pancreatic cancer; lung cancer; breast cancer; liver cancer; prostate cancer; ovarian cancer; cervical cancer; endometrial cancer; head and neck cancer; melanoma; testis cancer; urothelial cancer; renal cancer; stomach cancer; or carcinoid cancer. In a preferred embodiment the cancer is colorectal cancer; pancreatic cancer; lung cancer; breast cancer; liver cancer; prostate cancer; ovarian cancer; cervical cancer; endometrial cancer; head and neck cancer; or melanoma. In a particularly preferred embodiment the cancer is colorectal cancer; pancreatic cancer; lung cancer; breast cancer; or liver cancer. In a particularly preferred embodiment the cancer is a gastrointestinal cancer.

In one embodiment the tumor or the cells of the tumor are sensitive to neutrophil mediated ADCC or trogocytosis. In one aspect of the invention cells of the tumor are tested for sensitivity to neutrophil mediated ADCC or trogocytosis prior to treatment according to a method or purpose limited product claim according to the invention.

The extracellular membrane-bound antigen on cells is preferably a tumor antigen. It is preferably selected from the group consisting of: CD19, CD20, CD21, CD22, CD24, CD27, CD30, CD33, CD38, CD44, CD52, CD56, CD64, CD70, CD96, CD97, CD99, CD115, CD117, CD123, mesothelin, Chondroitin Sulfate Proteoglycan 4 (CSPG4), PD-L1 (CD274), Her2/neu (CD340), Her3, EGFR, PDGFR, SLAMF7, VEGFR1, VEGFR2, DR5, TF, GD2, GD3 or PTHR2. The extracellular membrane-bound antigen on cells is preferably selected from the group consisting of: CD19, CD21, CD22, CD24, CD27, CD30, CD33, CD38, CD44, CD52, CD56, CD64, CD70, CD96, CD97, CD99, CD115, CD117, CD123, mesothelin, Chondroitin Sulfate Proteoglycan 4 (CSPG4), PD-L1 (CD274), Her2/neu (CD340), Her3, EGFR, PDGFR, SLAMF7, VEGFR1, VEGFR2, DR5, TF, GD2, GD3, or PTHR2. In one embodiment the tumor or cells of the tumor are first tested for the presence of the indicated antigen prior to treatment according to a method or purpose limited product claim according to the invention.

### BRIEF DESCRIPTION OF THE DRAWINGS

FIG. 1: Inhibition of SIRPα enhances tumor eradication in a xenogeneic long-term in vivo model.

(A) Schematic overview of the in vivo xenogeneic model and the injection scheme (B-C) Long-term in vivo mouse model using human A431-SCR cells comparing the maternal cell line (B) to one with no expression of CD47 (A431-CD47KO) (C) Treatment started when mice had visual tumors at day 6 with either PBS (black line), a single iv injection of 50 μg cetuximab (red line) or an i.v. injection of 250 μg anti-EGFR-IgA2 followed by 4 i.p. injections due to the shorter half-life of IgA compared to cetuximab (blue line). Tumor outgrowth was measured with calipers and volume was calculated as length×width×height. Data shown are means±SEM from 1 experiment with 8 mice per group with (B) n=8, (C) n=8. Statistics performed are for day 17 using two-way ANOVA with Tukey's correction for multiple tests. ns=non-significant, *p<0.05, **p<0.01, ***p<0.001.

FIG. 2: Ba/F3 cells transfected with human HER2 (Ba/F3-HER2) or EGFR (Ba/F3-EGFR) are effectively killed by mouse neutrophils in vitro and in vivo when inhibiting SIRPα.

(A) Expression of HER2 and CD47 in Ba/F3-HER2 cells and EGFR and CD47 in Ba/F3-EGFR cells. (B) ADCC of trastuzumab or anti-HER2-IgA2 opsonized Ba/F3-HER2 cells by mouse neutrophils isolated from wildtype (NTg) or FcαR-transgenic (FcαR-Tg) mice combined without (white background) or with inhibition of SIRPα (grey background). (C) Same as (B), but with cetuximab or anti-EGFR-IgA2 opsonized Ba/F3-EGFR cells. (D) Inhibition of SIRPα is combined with trastuzumab or anti-HER2-IgA2 in an in vivo mouse experiment, showing the ratio of Ba/F3-HER2 and Ba/F3 cells in wildtype mice (PBS, Isotype+Tmab, anti-SIRPα+Tmab) or FcαR-transgenic mice (Isotype+anti-HER2-IgA2, anti-SIRPα+anti-HER2-IgA2). (E) Number of granulocytes (Ly6GC⁺/CD11b⁺) present in the peritoneal cavity at the end of the experiment in (D). (F) Overview of all cells present in the peritoneal cavity during the experiment shown in (D). (G) Inhibition of SIRPα is combined with anti-HER2-IgA2 and neutrophil depletion (Ly6G) in an in vivo mouse experiment, showing the ratio of Ba/F3-HER2 and Ba/F3 cells in wildtype mice (Isotype, anti-SIRPα, anti-SIRPα+anti-HER2-IgA2 (NTg)) or FcαR-transgenic mice (Isotype+anti-HER2-IgA2, anti-SIRPα+anti-HER2-IgA2, anti-SIRPα+anti-HER2-IgA2+Ly6G). Data shown for (B-C) are means±SEM pooled from 2 experiments with 2 mice per experiment with a total of n=4 individual mice. Data shown for (D-F) are means±SEM from 1 experiment with 6 mice per group with (A) n=6, (B) n=3-6, (C) n=6, (D) n=6 individual mice. Statistics shown for (B-C) are calculated by one-way ANOVA, with Dunnett's correction for multiple tests. Statistics shown for (D) and (G) are calculated by paired one-way ANOVA, with Sidak's correction for multiple tests. Statistics performed for (E) are calculated by one-way ANOVA, with Sidak's correction for multiple tests. ns=non-significant, *p<0.05, and **p<0.01, ***p<0.001.

FIG. 3: Primary sequence and modeling of the IgA1/IgA2.0 hybrid antibody.

A, alignment of primary sequences of the constant regions of hIgA1, IgA2m(1), and a IgA1/IgA2m(1) hybrid (hIgA2.0). Residues are numbered according to the myeloma IgA1 protein (Bur) scheme. Domain boundaries are indicated by vertical lines above the sequences. The following features are highlighted: light gray underlined residues are unique for IgA1, dark gray underlined asparagines are conserved N-glycosylation consensus sequences, and black underlined residues are unique for IgA2.0. B, the heavy chain of 225-IgA2.0 was modeled and illustrated in front and side view, with mutations marked. C, heavy chains of wild-type and mutant IgA2 were modeled. The resulting alignment indicates a different orientation of C241 in the heavy chains of IgA2-wt compared with IgA2.0, possibly due to the P221R mutation. D, focus on the tailpiece of 225-IgA2-wt (green, C471; red, Y 472) and IgA2.0 (red). Prediction and alignment of models were performed using I-TASSER; models were modified in 3D-Mol Viewer. E, illustration showing the amino acid sequence of the IgA2 heavy chain (UniProt reference no.: P01877). The highlighted amino acids depict amino acids that are subject to substitution in the IgA2.0 constant region. All or part of the underlined C-terminal amino acids can be deleted, to create an IgA3.0 constant region.

FIG. 4: Multiple sequence alignment of 3 CD47 antibody sequences.

The three antibodies are low (L), medium (M) or high (H) affinity for their target CD47 and differ significantly in their sequence, especially in their complementarity determining regions (CDRs). The sequences are derived from CD47 antibodies C47A8-CQ (low affinity), 5A3-M5 (medium affinity), and 2.3D11 (high affinity), documented in WO2014087248A2 (5A3-M5), EP2992089A1 Barbara Swanson et al Sorrento therapeutic (C47A8-CQ), and creative biolabs, Cat. No.: HPAB-0097-CN (2.3D11). The VH and VL sequences pasted in series, for alignment purposes but are not linked in in this way in the antibody.

FIG. 5: Transfection optimization and purification.

For production of antibodies, transfection conditions were designed in HEK293F cells (a). After choosing a condition for production, the antibody was purified first, by Kappa light chain affinity purification (b), followed by size exclusion chromatography (c). One representative picture is shown from the purification procedure.

FIG. 6: Binding specificity of CD47 antibodies.

Binding was tested in A431 (a) and CD47 knock out A431 (b) cell lines by flow-cytometry. The three antibodies (L,M,W) show specific binding, with varying affinity (c).

FIG. 7: Antibody dependent cell mediated cytotoxicity (ADCC).

ADCC assay to determine killing activity of four different CD47 antibodies, as described above and B6H12, a mouse IgG1 mAb as a positive control for NK cell mediated killing. For the ADCC assay with either PBMCs (a) or PMNs (b), the effector:target cell ratio was 100:1 and 40:1 respectively. Cytotoxicity was measured after 4 hrs. n=3 replicates ±SEM.

FIG. 8: Neutrophil-mediated ADCC-induced cell lysis in Daudi cells with anti CD47 and anti CD20 combination treatment.

ADCC assay to determine the killing activity of anti-CD47 antibodies (clone 2.3D11) and anti-CD20 antibodies (variable region of Obinutuzumab) of the IgG or IgA3.0 isotype, as well as combinations of anti-CD47 and anti-CD20 antibodies. Daudi cells were incubated with effector cells and either 10, 1 or 0.1 μg/ml (high-med-low concentration) anti-CD20 antibody and/or 20, 2, 0.2 μg/mL anti-CD47 antibody (high-med-low). Obi is an anti-CD20 antibody with the variable domain of Obinutuzumab.

FIG. 9: Neutrophil-mediated ADCC-induced cell lysis in Ramos cells with anti CD47 and anti CD20 combination treatment.

(a) The three CD47 antibodies of FIGS. 6 and 7 were tested at concentrations of 0, 0.2, 2 and 20 μg/ml. The CD20 antibody was tested at concentrations of 0, 0.1, 1 and 10 μg/ml. (b) is an experiment similar to (a). In (a) the CD20 antibody had an IgA2.0 constant part, whereas in (b) IgA3.0 anti-CD20 antibodies were used. In both experiment (a) and (b) killing was enhanced in anti-CD47 and anti-CD20 combination treatment. Cell lysis was measured after 4 hr. n=3 replicates; ±SEM. Obi is an anti-CD20 antibody with the variable domain of Obinutuzumab.

FIG. 10: Neutrophil-mediated ADCC-induced cell lysis in Ramos cells with anti CD47 and anti CD2 combination treatment.

Neuroblastoma cancer cell lines SH-Sy5y (FIGS. 10A and 10D), SKNFI (FIGS. 10C and 10E) and LAN5 (FIG. 10B) were incubated with anti-GD2 antibodies having either an IgA or IgG isotype and in absence or presence of anti-CD47 antibody. Whole leukocytes (FIG. 10A-C) or peripheral mononuclear cells (PMNs) (FIGS. 10D and 10E) were used as effector cells. In conditions wherein the cancer cells were incubated with a single antibody, 10, 1, or 0.1 μg/ml antibody was used. In combination therapy of anti-GD2 and anti-CD47 antibodies, 10, 1, or 0.1 μg/ml IgA anti-GD2 antibody was combined with 20, 2, or 0.2 μg/ml anti-CD47 antibody. The methods applied for determining ADCC are the same as for FIGS. 7, 8 and 9. Cell lysis was measured after 4 hr. n=3 replicates; ±SEM.

### EXAMPLES

### Example 1

Results

IgA Induces Cytotoxicity of A431 and Ba/F3 Cancer Cells when Inhibiting CD47-SIRPα Interactions in In Vivo Xenogeneic and Syngeneic Mouse Models.

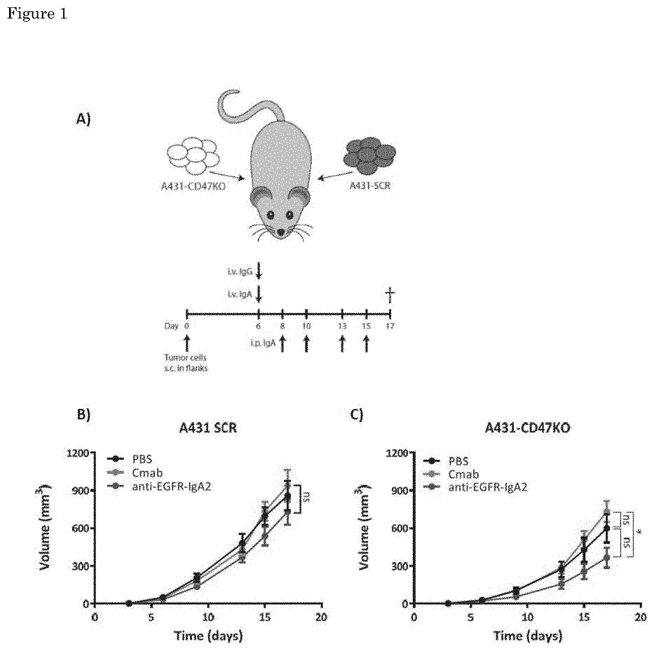

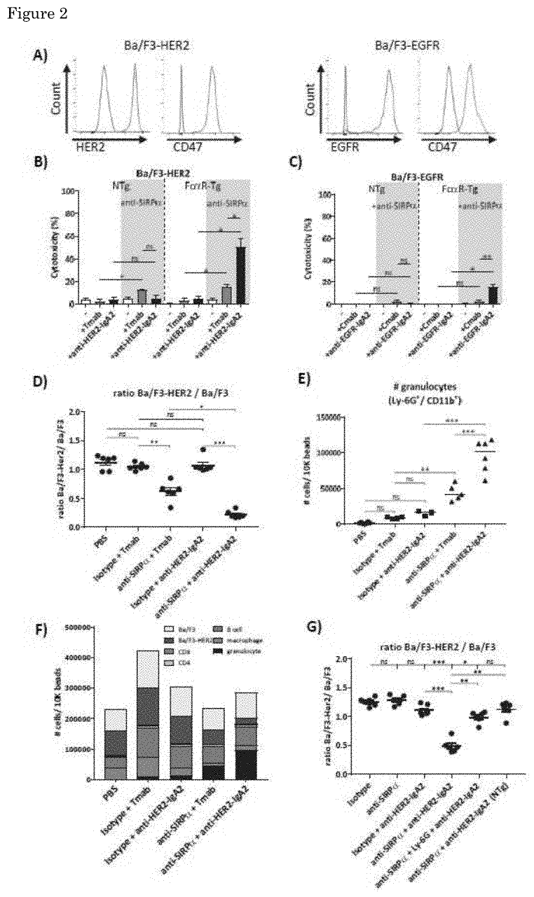

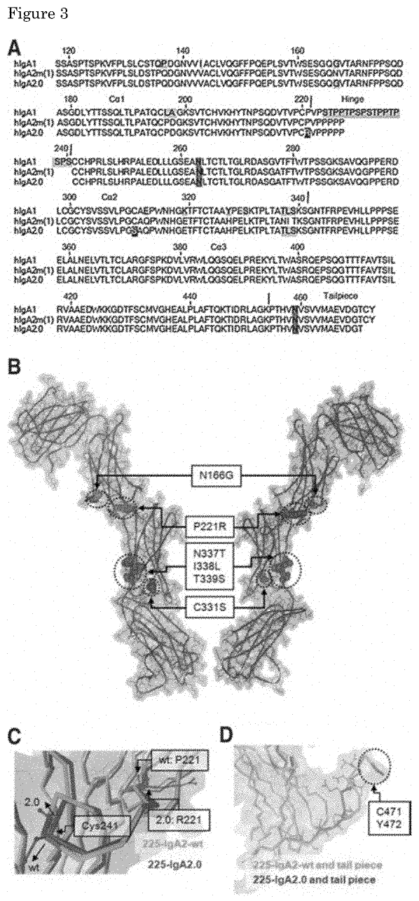

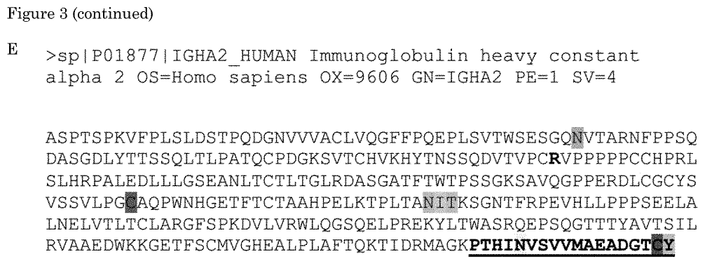

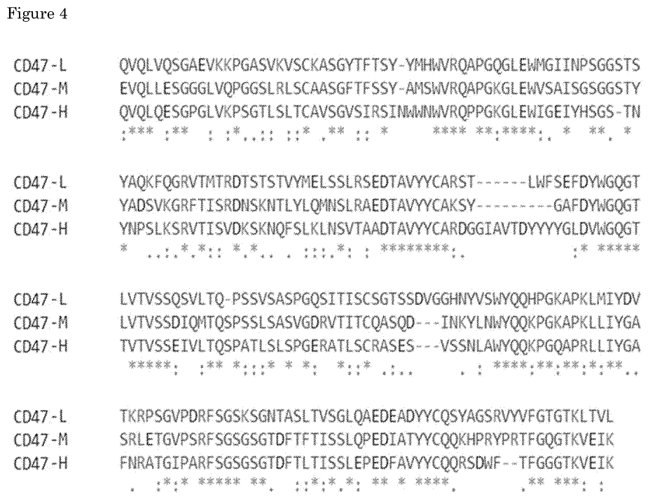

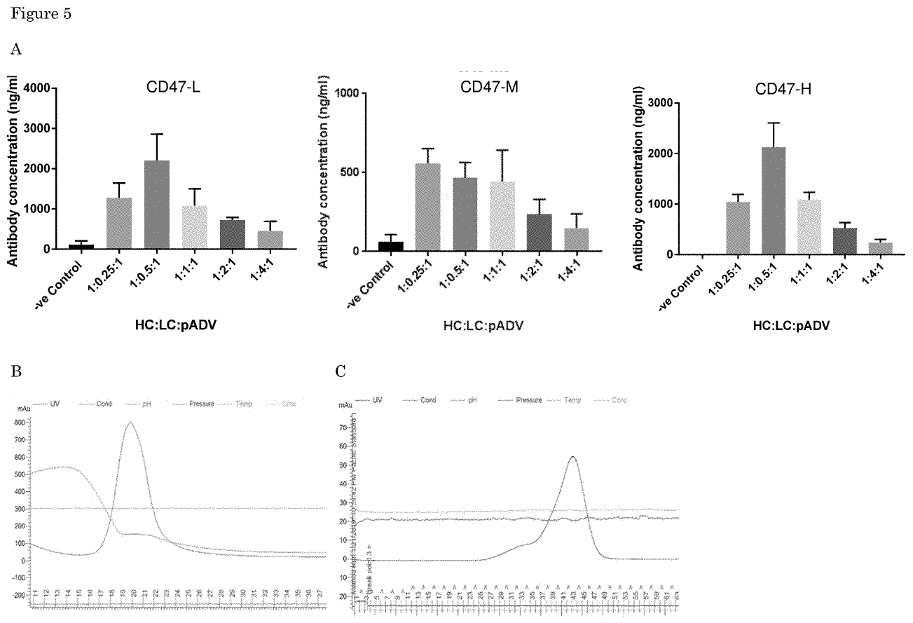

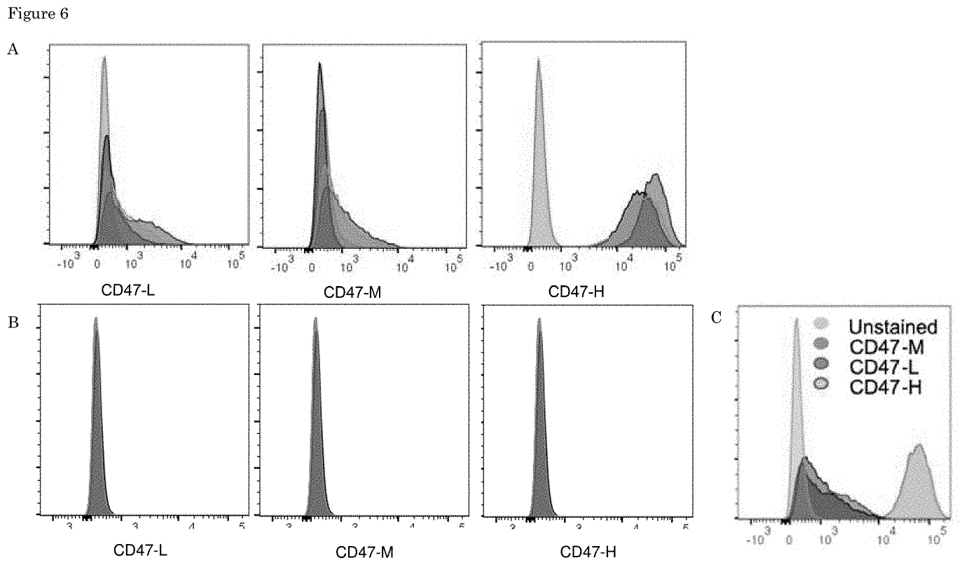

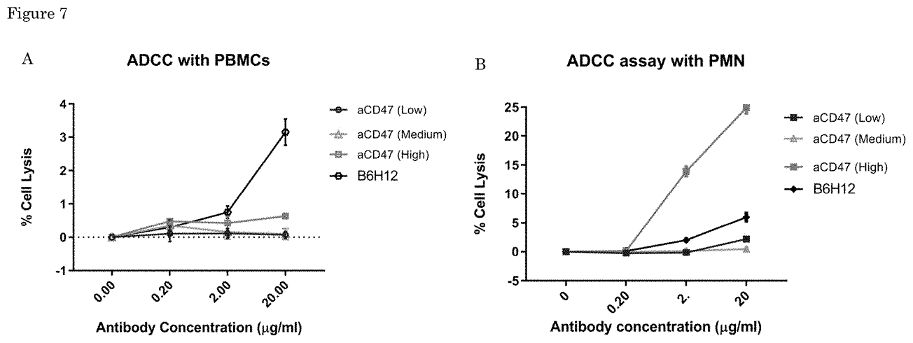

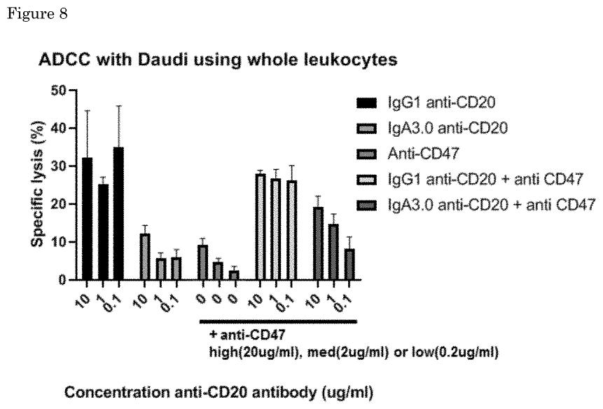

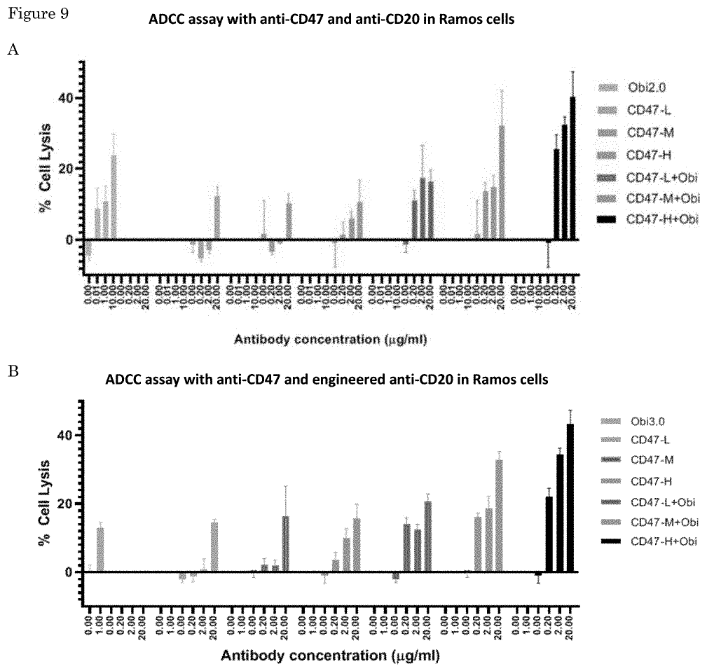

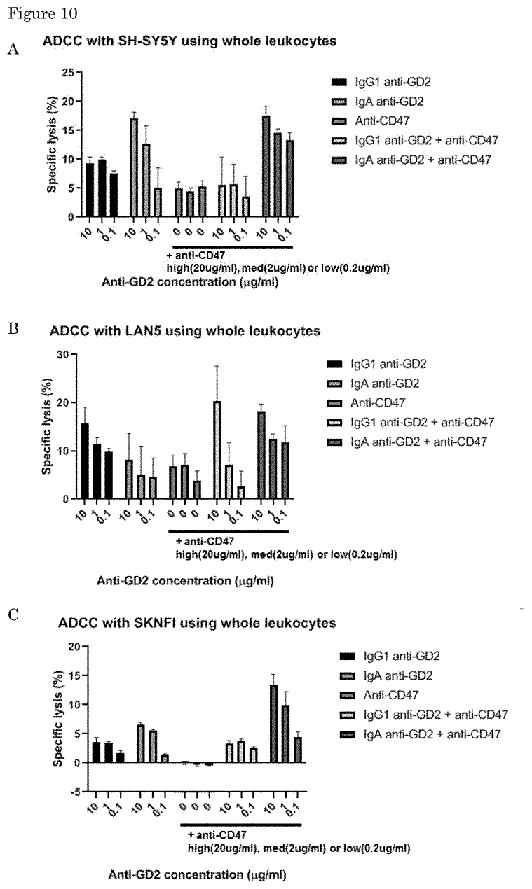

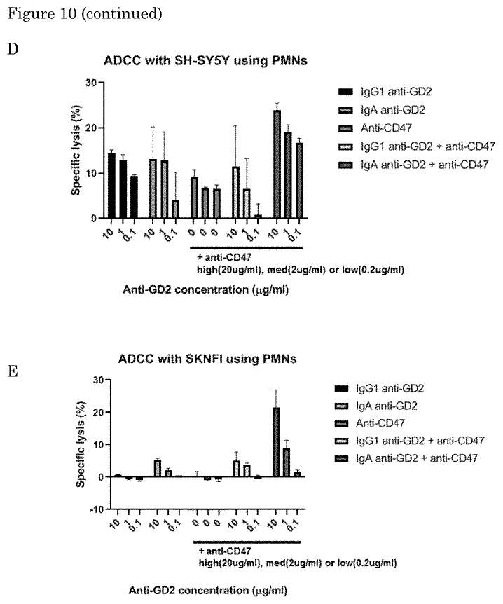

The effect of blocking CD47-SIRPα interactions in combination with IgA therapeutic antibody was evaluated in an in vivo setting. Mice were used expressing human FcαRI (Boross et al, 2013 EMBO Mol Med 5: 1213-26) on a SCID background. A long-term xenogeneic in vivo mouse model with the human epidermoid cell line A431 was investigated. In this particular model we compared the tumor growth, in the same mouse, of a CD47 expressing A431 cell line to one where we removed expression of CD47 by CRISPR/Cas9 interference (A431-CD47KO). Groups of mice were treated with either PBS, cetuximab or anti-EGFR-IgA2 as indicated (FIG. 1A). After 17 days, tumor volume was significantly reduced only of the CD47KO A431 tumors after anti-EGFR-IgA2 treatment compared to cetuximab (FIG. 1B, C).

To examine the role of neutrophils as effector cells in vivo, we made use of a syngeneic mouse model in mice expressing human FcαRI (Boross et al, 2013 EMBO Mol Med 5: 1213-26) in combination with the anti-mouse SIRPα specific antibody MY-1 to block the interaction between CD47 and SIRPα (Yanagita et al; 2017 JCI insight 2(1):e89140). To determine the capacity of mouse neutrophils to perform ADCC in this system, we first isolated mouse neutrophils from bone marrow from FcαRI transgenic and wild type mice. The mouse pro-B cell line Ba/F3, which does not express mouse SIRPα, was used as target, either expressing human HER2/neu or EGFR, in combination with IgA2/IgG anti-HER2/neu or anti-EGFR accompanied by MY-1 (FIG. 2A). No antibody-dependent neutrophil-mediated killing of both target lines was observed in the presence of an intact CD47-SIRPα signaling axis. However, the IgA2 variant of especially the anti-HER2/neu therapeutic antibody significantly enhanced ADCC by neutrophils expressing FcαR, which could be further increased by SIRPα checkpoint blockade for both HER2/neu and EGFR-expressing Ba/F3 cells (FIG. 2B, C). These results show that the use of IgA therapeutic antibodies resulted in significantly higher ADCC compared to IgG in this setup, specifically after blocking SIRPα/CD47 signaling by the blocking antibody MY-1, which enhanced killing of HER2/neu and EGFR-expressing tumor cells by murine neutrophils.

Next, we assessed the effect of SIRPα block on IgA therapy in a syngeneic mouse model as previously described (Boross et al, 2013 EMBO Mol Med 5: 1213-26). We compared therapeutic antibody of either anti-HER2 IgG or IgA subclass in the presence or absence of CD47/SIRPα blockade. Fluorescent Ba/F3 and Ba/F3-HER2 cells were injected in the peritoneal cavity and combined with therapeutic antibodies in the presence or absence of CD47/SIRPα inhibition. Saturation of the SIRPα receptor with MY-1 on both macrophages and neutrophils was confirmed using flow cytometry (data not shown). Comparable to the in vitro setting, blocking CD47/SIRPα in vivo by MY-1, led to a significant and substantial increased reduction of tumor load compared to the use of either IgG or IgA therapeutic antibodies alone (FIG. 2D). Of interest, the antibody-mediated reduction of the tumor load was accompanied by a significant influx of granulocytes in the condition where MY-1 was combined with therapeutic antibody. This neutrophil influx was most evident when using anti-HER2-IgA2 compared to trastuzumab (FIGS. 2E, F). No alteration in the influx of other leukocyte populations was detected under this condition, apart from a small decrease in macrophages (FIG. 2F). The data show that blockade of CD47-SIRPα signaling increases the therapeutic potency of anti-HER2-IgA2 in a syngeneic in vivo mouse model.

That neutrophils are the effector cell population responsible for the killing of the Ba/F3-HER2 cells in the in vivo mouse model, was shown by depleting neutrophils by the use of anti-Ly6G antibody. This depletion did not result in significant changes in other leukocyte populations (data not shown). When neutrophils were depleted from the mice, only limited ADCC occurred when using both anti_EGFR-IgA2 and MY-1 (FIG. 2G), indicating that indeed neutrophils are involved in the cytotoxic effect in this mouse tumor model. The results show that neutrophils are important effector cells that can successfully be recruited for therapeutic clearance by anti-tumor antigen antibodies and that the therapeutic effect is increased by inhibiting CD47-SIRPα signaling.

The results show that IgA-mediated anti-tumor therapy can be restricted by CD47-SIRPα interactions in vitro and in vivo. This is inferred from both short-term syngeneic and long-term xenogeneic mouse models described here. Importantly, this restriction can be therapeutically overcome by CD47 or SIRPα blocking antibodies.

Materials and Methods

Cells and Culture

Cell lines were from ATCC (A431 and Ba/F3) were kept in culture according the suppliers' recommendations.

A431-CD47KO cell lines were generated by lentiviral transduction of pLentiCrispR-v2-CD47KO (pLentiCrispR-v2 was a gift from Feng Zhang (Addgene plasmid #52961)), using 5′ cagcaacagcgccgctacca 3′ as the CD47 CrispR target sequence. Transduced cells were selected with 1 μg/mL of puromycin, followed by limiting dilution. A clone lacking CD47 expression was selected by FACS staining. A431 cells expressing HER2/neu (A431Her2Neu) were generated by retroviral transduction, followed by positive selection based on puromycin resistance, as previously described (Brandsma et al 2015: Cancer Immunol Res 3(12): 1316-24).

Ba/F3 cells expressing EGFR were transfected with WT EGFR (upstate) and EGFR expressing clones were selected using neomycin. Ba/F3 cells expressing HER2/neu were generated by retroviral transduction, followed by positive selection using puromycin resistance and limiting dilution.

Neutrophil Isolation

Mouse neutrophils were isolated from mouse bone marrow as follows: rat anti mouse-CD16/CD32 (BD Pharmingen, clone 2.4G2) was incubated in 1 mL MACS-buffer (containing phosphate-buffered saline (PBS), pH 7.2, 0.5% bovine serum albumin (BSA), and 2 mM EDTA) at a concentration of 25 μg/mL for 20 minutes on ice. After incubation, anti-Ly6G-APC (clone 1A8, BD Biosciences) was directly added at a concentration of 1 μg/mL for 30-45 minutes on ice. Finally, 20 μL anti-APC MicroBeads (Miltenyi Biotec) were added per 10⁶ cells and put for 60 minutes on ice. After isolation, mouse neutrophils were cultured overnight at a concentration of 5*10⁶ cells/mL, in the presence of 50 ng/mL mouse IFNγ (PeproTech) and 10 ng/mL clinical grade G-CSF (Neupogen), after which the cells were used in assays the following day.

Antibodies and Reagents

IgG trastuzumab (Roche), IgG cetuximab (Merck KGaA), anti-HER2-IgA, anti-CD20-IgA2 and anti-EGFR-IgA2) were generated as described (Dechant 2007: J. Immunol. 179(5):2936-43, Meyer 2015 MABs 8(1):87-98) and used at a final concentration of 0.5 μg/mL, unless stated otherwise.

To block mouse SIRPα we used the rat IgG2 antibody MY-1, generously gifted to us by the group of Takashi Matozaki (University of Kobe, Japan), at a final concentration of 10 μg/mL in our in vitro studies (Yanagita et al; 2017 JCI insight 2(1):e89140))(Yanagita et al; 2017 JCI insight 2(1):e89140).

Mice

Experiments for the Ba/F3 peritoneal model were performed with 12- to 38-wks old male and female human FcαR transgenic g) mice which were generated at UMC Utrecht (Van Egmond et al 1999: Blood 93(12)4387-94) and backcrossed on a Balb/cByJRj background (Janvier, France) and maintained in hemizygous breeding. Transgene negative (NTg) littermates were used as control mice. Experiments for the A431 in vivo model were performed with 17- to 70-wks old female human FcαR transgenic (Tg) mice which were generated at UMC Utrecht (Van Egmond et al 1999: Blood 93(12)4387-94) and backcrossed on a SCID background (CB17/1CR-Prkdc_{SCID}/Crl, Charles River) and maintained in hemizygous breeding. All mice were bred at the specific pathogen-free facility of the Central Animal Laboratory of Utrecht University, all experiments were approved by the central committee animal experiments (license #AVD115002016410). The Ba/F3 peritoneal model was described previously (Boross et al, 2013 EMBO Mol Med 5: 1213-26). Briefly, Ba/F3-HER2 and Ba/F3 cells were labelled with respectively 2 μM or 10 μM CT violet (Invitrogen, Thermofisher) for 15 min at room temperature and mixed thereafter at 1:1 ratio. In total 1×10⁷ cells were injected per mouse intraperitoneally in 200 μL PBS. 200 μg My-1 was used to block mouse SIRPα in vivo which was injected 2 days before tumor cell injection and mixed with the treatment consisting of anti-HER2-IgG1 or anti-HER2-IgA2 (100 μg) injected intraperitoneally directly after the injection of tumor cells. Sixteen hours later the mice were euthanized, the peritoneum washed with PBS containing 5 mM EDTA, the absolute number of Ba/F3-HER2 and Ba/F3 determined by flow cytometry using TruCount tubes (BD biosciences) and the ratio of Ba/F3-HER2 and Ba/F3 was calculated. Effector cells in the peritoneum were determined using specific antibodies and there relative amount was related to constant amount of beads (Invitrogen).

For the A431 in vivo model mice were injected with 5×10e5 A431-CD47KO cells on the right flank, 5×10⁵ A431 scrambled (A431-SCR) control cells were injected in the same mouse on the left flank. On day 6 all the mice had visual A431-CD47KO and A431-SCR control tumors and i.v. treatment started with a single injection of 50 μg IgG cetuximab or 250 μg anti-EGFR-IgA2. Anti-EGFR-IgA2 has a shorter half-life compared to cetuximab therefore anti-EGFR-IgA2 treatment was continued by i.p. injections on days 8, 10, 13, 15 and 17 (250 μg). Tumor outgrowth was measured twice a week with calipers and volume was calculated as length×width×height.

Flow Cytometry

Effector cells in the peritoneum were determined after incubation with 5% normal mouse serum (Equitech-bio) for 45 min on 4-7° C. Subsequently, the following fluorescently labelled antibodies were used for 45-60 min on 4-7° C. to stain for different effector cells types: B220 (RA3-6B2) C, I-A/I-E (M5/114.15.2), CD8 (53-6.7), Ly-6G (1A8), CD45 (30-F11), CD4 (RM4-5), F4/80 (BM8) (Biolegend) and CD11b (ml/70) (BD biosciences).

After excluding Ba/F3 cells from the analysis granulocytes were identified as Ly-6G+/CD11b+ and F4/80-, macrophages were identified as F4/80+/CD11b+ and Ly-6G-lymphocytes were analyzed by first excluding Ba/F3 cells, F4/80+/CD45+ macrophages and dead cells (7AAD+) followed by CD45 selection were B cells were identified as B220+/I−A/I−E+ and T cells as being CD4+ or CD8a+. Saturation of SIRPα in vivo was determined by comparing staining for the injected MY-1 with anti-rat Ig (BD biosciences) with ex vivo added MY-1 or isotype control and anti-rat Ig both followed by staining for macrophages and granulocytes. Measurements were performed on a FACSCantoII (BD biosciences), data were analyzed using FACS Diva software (BD biosciences).

For the CD47-beads binding assay goat anti-human Alexa647 IgG (H+L) (Invitrogen) was used at a concentration of 20 μg/mL.

Cell lines were analyzed for expression of HER2 using trastuzumab, EGFR using cetuximab, human CD47 using B6H12 (15), murine CD47 using miap301 (eBioscience), murine SIRPα using MY-1 (26).

Data Analysis and Statistics

Statistical differences between two groups were tested using (paired) t test; multiple comparisons were tested using two-way ANOVA with Tukey's correction for multiple tests or one-way ANOVA-test followed by Sidak or Dunett post-hoc test for correction of multiple comparison by GraphPad Prism (GraphPad Software).

Discussion

We show here for the first time that neutrophil-mediated cytotoxicity of cancer cells by IgA antibodies against Her2/neu, EGFR and CD20 is subject to inhibition by CD47-SIRPα, both in vitro and in vivo. This new finding shows that inhibition of the CD47-SIRPα axis further enhances IgA mediated anti-tumor effects with neutrophils as effector cells. A number of CD47 antibodies and SIRPα-Fc proteins are currently being tested in phase PII clinical trials in combination with IgG therapeutic antibodies (www.clinicaltrials.gov identifiers: NCT02216409; NCT02678338, NCT02641002; NCT02367196, NCT02890368; NCT02663518, NCT02953509)(20).

Here, we provide proof that neutrophils are important effector cells against the IgA antibody-opsonized cancer cells in our syngeneic in vivo model. This is in contrast to our earlier work, when macrophages were the dominant effector cells eliminating the IgA-opsonized tumor cells in another short in vivo model using Ba/F3 cells overexpressing EGFR (Boross et al, 2013 EMBO Mol Med 5: 1213-26). In this particular model, besides the fact that a different therapeutic antibody targeting a different tumor antigen is used with different antigen expression levels, the CD47-SIRPα axis is intact. This likely contributed to the different effector cell population being activated and recruited against the antibody-opsonized cancer cells. The invention shows that both neutrophils and macrophages can mediate cytotoxicity by IgA anti-tumor antibodies.

Recently, checkpoint inhibition of the CD47-SIRPα axis was found to increase effective T-cell responses against the tumor by activating CD8⁺ T cells and suppressing CD4⁺ T cells (Tseng et al, 2013: Proc. Natl. Aced. Sci. USA 110:11103-8). Although the exact mechanism by which CD47-SIRPα checkpoint inhibition stimulates a cytotoxic T cell response against the tumor is unknown, it would be feasible to involve enhanced macrophage-mediated antigen presentation in response to an increased uptake of tumor material (Tseng et al, 2013: Proc. Natl. Acad. Sci. USA 110:11103-8), or even an increase and contribution of neutrophil antigen presentation (Vono et al, 2017: Blood 129(14):1991-2001). Combining an IgA-based antibody therapy against cancer with inhibition of CD47-SIRPα interactions can lead to an efficient adaptive response in later stages of tumor therapy.

Thus far, an important drawback of using IgA anti-cancer antibodies clinically is the short half-life of IgA antibodies in vivo (Boross et al, 2013 EMBO Mol Med 5: 1213-26). Currently, we and others have found ways to extend the life-span of IgA antibodies, e.g. by glyco-engineering strategies (Lohse et al, 2016: Cancer Res. 76(2):403-17; Rouwendal et al, 2016: MAbs 8(1):74-86) or by increasing the binding to the neonatal Fc-receptor FcRn with the help of antibody engineering (Borrok et al, 2015: MAbs 7(4):743-51; Meyer S et al, 2016 MAbs 8(1):87-98; Li B et al, 2017: Oncotarget. 8(24):39356-66), making their use in the clinic even more effective.

Also contemplated in the present invention is the use of so-called IgGA-antibodies, such an antibody that can bind both Fey-receptors, FcαR and FcRn. Such an IgGA antibody has a half-life comparable to IgG antibodies, and is able to engage NK cells, macrophages, monocytes, and neutrophils very effectively both in vitro as well as in vivo (Borrok et al, 2015: MAbs 7(4):743-51; Li B et al, 2017: Oncotarget.

8(24): 39356-66).

### Example 2

Results

In Combination with an IgA Isotype Antibody Recognizing a Tumor Antigen, Anti-CD47 Antibodies Show a Dose-Dependent Enhanced Killing of Both Daudi and Ramos Cells.

The effect of blocking CD47-SIRPα interactions using anti-CD47 antibodies in combination with an IgA therapeutic antibody was evaluated in an in vitro setting. To this end, three different anti-CD47 antibodies (FIG. 4) were selected for expression and purification. HEK293F cells were transfected with expression vectors for the respective antibodies in different conditions (FIG. 5A) and the best condition was chosen for large scale production. The antibody was purified from the cell culture supernatant using kappa light chain affinity purification (FIG. 5B), followed by size exclusion chromatography (FIG. 5C). The three antibodies showed different affinity for CD47 (low-medium-high) (FIGS. 6A and C), but all three antibodies bind CD47 with high specificity (FIG. 6B).

Next, ADCC-activity of the three antibodies was tested using either PMNs or PBMCs as effector cells, and Daudi cells as target cells. Incubation of cancer cells with anti-CD47 antibodies alone resulted in cell lysis (FIGS. 7A and B), which was dependent on the concentration of the antibody. Cell lysis was most evident when using PMNs as effector cells (FIG. 7B). In this case, the anti-CD47 having a high affinity induced the highest percentage of cell lysis. The B6H12 mIgG1/k clone does not provoke ADCC as it is a mIgG1/k antibody that does not act on neutrophil activation. Hence, only a signal is observed in PBMC.

Subsequently, we compared ADCC induced by anti-CD20 antibodies having either an IgG or IgA isotype with the anti-CD47 antibody having a high affinity. Both CD47/SIRPα checkpoint inhibition as well anti-CD20 antibodies having an IgA3.0 constant region separately induced cell lysis dose dependently (FIG. 8). However, upon combination of these two, the percentage of lysed cells was significantly increased (FIG. 8).

Similar results were obtained in two experiments wherein Ramos cells were used as target cells (FIGS. 9A and 9B). Both anti-CD20 antibodies having an IgA2.0 or IgA3.0 constant region, as well as anti-CD47 antibodies induced cell lysis. Of note, anti-CD47 antibodies having a high affinity seemed to do so more efficiently. Upon combination of CD47/SIRPα blockade with an anti-CD20 antibody having an IgA2.0 or 3.0 constant region, cell lysis increased dose-dependently. Also in combination with anti-CD20 antibodies, the percentage of cell lysis that was induced correlated with the affinity of the anti-CD47 antibody.

For three different neuroblastoma cell lines (SH-SY5Y, SKNFI, LAN5), we observed a dose dependent effect of incubation with anti-GD2 antibodies (both IgG and IgA) (FIGS. 10A-E). Anti-CD47 induced ADCC only in SH-SY5Y and LAN5 cells. Surprisingly, upon combination of anti-CD47 antibodies with anti-GD2 antibodies having an IgA isotype, an additive effect was observed. This did not seem to be the case for the same combination wherein the anti-GD2 antibodies had an IgG isotype. These effects were independent of the target cells used, as results were comparable between experiments wherein PMNs or whole leukocytes were used.

Materials and Methods

Cells and Culture

Cell lines were acquired from ATCC (A431, Daudi, Ramos) and cultured in RPMI culture medium containing RPMI-1640+HEPES+glutamine (Invitrogen) supplemented with 10% fetal calf serum (FCS) and 100 U/mL penicillin and 100 μg/mL streptomycin (lx P/S; Life Technologies at 37° C. and 5% CO₂. A431-CD47KO cell lines were generated by lentiviral transduction of pLentiCrispR-v2-CD47KO (pLentiCrispR-v2 was a gift from Feng Zhang (Addgene plasmid #52961)), using 5′ cagcaacagcgccgctacca 3′ as the CD47 CrispR target sequence. Transduced cells were selected with 1 μg/mL of puromycin, followed by limiting dilution. A clone lacking CD47 expression was selected by FACS staining.

FreeStyle™ HEK293F cells (Invitrogen) were cultured in FreeStyle™ 293 expression medium (Invitrogen) at 37° C. and 8% CO₂ on an orbital shaker. PBMC and PMN were isolated from healthy individuals (MiniDonorDienst UMC Utrecht) by Ficoll separation (GE healthcare).

ADCC Assay

Cancer cell lines were labelled with 100 μCi ⁵¹Cr (Perkin Elmer) per 1×10\hat{ }6 cells for 3 hours at 37° C. and 5% CO₂. Next, cells were washed in PBS and seeded in a 96-wells U-bottom plate. 5×10³ cells per well were incubated for 4 hours with effector cells and therapeutic antibody/antibodies at 37° C., 5% CO₂. The ratio effector cells:target cells was either 40:1 (PMN) or 100:1 (PBMC). The antibody concentration in conditions with a single antibody was 0.1, 1, or 10 μg/ml. In conditions wherein anti-CD47 antibodies were combined with anti-CD20 antibodies, the concentration of anti-CD20 antibodies was 0.1, 1 or 10 μg/mL, and anti-CD47 20 μg/ml. After incubation, the supernatant was harvested an analyzed for radioactivity using a gamma counter (Wallac). The maximal number of count per minute (cpm), or total cpm was determined by incubation of the target cells with 2.5% Triton X-100 (Roche Diagnostics). The number of spontaneous cpm was determined by incubation of target cells in absence of effector cells. The percentage of cytotoxicity was calculated using the following formula: [(experimental cpm−spontaneous cpm)/(determined as follows: % specific lysis=(count experiment−minimal lysis)/(total cpm−spontaneous cpm)]×100%. All conditions were measured in triplicate.

Antibodies and Reagents

CD47 mAb clone B6H12 was from eBioscience, mouse IgG1/k. As CD47 antibodies a low affinity (C47A8-CQ), medium affinity, (5A3-M5), and high affinity (2.3D11) antibodies were chosen, documented in WO2014087248A2(5A3-M5), EP2992089A1 Barbara Swanson et al Sorrento therapeutic (C47A8-CQ), and creative biolabd, Cat. No.: HPAB-0097-CN (2.3D11)

The low-medium-high affinity anti-CD47 antibodies were generated by cloning the variable regions in to Lonza expression vectors. Antibodies were purified using select columns followed by size exclusion columns (GE healthcare).

For targeting GD2, anti-GD2 antibodies having the variable domain of clone ch14.18 were used. CD20-IgA2 was generated as described in: Dechant 2007: J. Immunol. 179(5):2936-43, and Meyer 2015 MABs 8(1):87-98.

Antibodies having an IgA2.0 constant region were obtained by introducing the following substitution and deletions: N45.2G, P124R, C92S, N120T, I121L, T122S, deletion of C147 and deletion of Y148, numbering according to IMGT scheme. Additionally, an N135Q mutation (numbering according to IMGT scheme) can be introduced. In order to create an IgA3.0 constant region, 3-20 C-terminal amino acids can be removed (see FIG. 3E).

Flow Cytometry

Binding of the different anti-CD47 antibodies was analyzed on 10⁵ cells per condition. Cells were stained with 10, 1 or 0.1 μg/ml antibody for 30 minutes at room temperature, washed, and subsequently stained 1:200 for 30 minutes with an anti-mouse, fluorophore-labelled secondary antibody for 30 minutes. Hereafter, cells were washed an fixed. Fluorescence was acquired using a FACS Canto II (BD Bioscience), and data was analyzed using FlowJo software (Treestar).

Data Analysis and Statistics

Standard error of the mean (SEM) was calculated using GraphPadPrism.

Discussion

Here, we describe for the first time that inhibition of the CD47/SIRPα checkpoint using an anti-CD47 antibody when combined with a second therapeutic antibody having an IgA isotype results in increased ADCC. This increase is both dose dependent and affinity dependent (FIG. 7B and FIG. 9), and was observed in both Daudi and Ramos target cells (FIGS. 7 and 9 respectively) as well as a panel of three different neuroblastoma cell lines (FIGS. 10A-E).

The findings in the present invention show that IgA cancer immunotherapy is inhibited by CD47-SIRPα signaling. Targeting the CD47-SIRPα signaling can be used to increase the anti-tumor efficacy of IgA therapeutic antibodies, preferably in cancer treatments.

1. A method of stimulating neutrophil-mediated killing of CD47 expressing cells comprising contacting neutrophils with cells that express CD47 and another extracellular membrane-bound antigen in the presence of a first and a second binding moiety, wherein said first binding moiety specifically binds a myeloid IgA receptor (CD89) and said antigen, and wherein said second binding moiety specifically binds CD47 and/or SIRPα and blocks CD47 mediated signaling of SIRPα in said neutrophil.


SEQUENCE LISTING (10 sequences)

SEQ ID NO: 1 - protein, 6 aa, Homo sapiens
VDGTCY

SEQ ID NO: 2 - protein, 4 aa, Artificial Sequence
  mutated IgA2m(1) sequence
VDGT

SEQ ID NO: 3 - protein, 355 aa, Homo sapiens
SSASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 4 - protein, 342 aa, Homo sapiens
SSASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 5 - protein, 340 aa, Artificial Sequence
  hIgA2.0
SSASPTSPKVFPLSLDSTPQDGNVVVACLVQGFFPQEPLSVTWSESGQGVTARNFPPSQDASGDLYTTSSQLTLPATQCPDGKSVTCHVKHYTNPSQDVTVPCRVPPPPPCCHPRLSLHRPALEDLLLGSEANLTCTLTGLRDASGATFTWTPSSGKSAVQGPPERDLCGCYSVSSVLPGSAQPWNHGETFTCTAAHPELKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGT

SEQ ID NO: 6 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTYAVTSILRVAAEDWKKGETFSCMVGHEALPLAFTQKTIDRMAGKPTHINVSVVMAEADGTCY

SEQ ID NO: 7 - protein, 229 aa, Artificial Sequence
  CD47-L
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARSTLWFSEFDYWGQGTLVTVSSQSVLTQPSSVSASPGQSITISCSGTSSDVGGHNYVSWYQQHPGKAPKLMIYDVTKRPSGVPDRFSGSKSGNTASLTVSGLQAEDEADYYCQSYAGSRVYVFGTGTKLTVL

SEQ ID NO: 8 - protein, 224 aa, Artificial Sequence
  CD47-M
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSYGAFDYWGQGTLVTVSSDIQMTQSPSSLSASVGDRVTITCQASQDINKYLNWYQQKPGKAPKLLIYGASRLETGVPSRFSGSGSGTDFTFTISSLQPEDIATYYCQQKHPRYPRTFGQGTKVEIK

SEQ ID NO: 9 - protein, 231 aa, Artificial Sequence
  CD47-H
QVQLQESGPGLVKPSGTLSLTCAVSGVSIRSINWWNWVRQPPGKGLEWIGEIYHSGSTNYNPSLKSRVTISVDKSKNQFSLKLNSVTAADTAVYYCARDGGIAVTDYYYYGLDVWGQGTTVTVSSEIVLTQSPATLSLSPGERATLSCRASESVSSNLAWYQQKPGQAPRLLIYGAFNRATGIPARFSGSGSGTDFTLTISSLEPEDFAVYYCQQRSDWFTFGGGTKVEIK

SEQ ID NO: 10 - DNA, 20 bp, Artificial Sequence
  CD47 Crispr target sequence
cagcaacagcgccgctacca